(19) United States
(12) Patent Application Publication
(10) Pub. No.: US 2008/0020409 A1
(43) Pub. Date: Jan. 24, 2008

(54) Analytical Platform and Method for Generating Protein Expression Profiles of Cell Populations

(76) Inventors: Michael Pawlak, Nehren (DE); Eginhard Schick, Rheinfelden (DE); Peter Oroszlan, Basel (CH)

Correspondence Address: NORRIS, MCLAUGHLIN & MARCUS, PA, 875 THIRD AVENUE, 18TH FLOOR, NEW YORK, NY, 10022, US

(21) Appl. No.: 10/598,418

(22) PCT Filed: Mar. 3, 2004
(86) PCT No.: PCT/EP04/02127

§ 371 Date: Dec. 11, 2006

(51) Int. Cl. G01N 33/574; C12M 1/34; C40B 30/04; G01N 33/567; G01N 33/569

(52) U.S. Cl. 435/7.23; 435/288.7; 435/7.2; 435/7.21; 435/7.32; 506/9

(57) ABSTRACT

The present invention is related to analytical platforms and methods performed therewith for generating qualitative and/or quantitative protein expression profiles, in particular differential protein expression profiles, of cell populations comprising: generating lysates of one or more populations of cells, the lysates comprising a plurality of proteins expressed by the respective cell populations, providing an essentially planar solid support, depositing at discrete sites small quantities of the cell lysates, in diluted or undiluted form directly on said solid support or on an adhesion-promoting layer applied on said solid support, thereby creating one or more one- or two-dimensional arrays of discrete measurement areas on said solid support, applying a number of binding reagents as specific binding partners for the proteins contained in cell lysates in discrete measurement areas and to be detected and, if adequate, one or more detection reagents on said one or more arrays of measurement areas, the binding reagents and the detection reagents being applied sequentially or in a single addition-step, after binding of the detection reagents to the binding reagents, to the one or more arrays of discrete measurement areas for e.g. global analysis of signaling pathways or screening antibody sets/libraries against protein targets for best specificity, selectivity and affinity, and measuring and recording optical signals emanating from said one or more arrays of discrete measurement areas in a locally resolved manner, wherein said essentially planar solid support is non-porous and an optionally applied adhesion-promoting layer has a thickness of less than 1 μm.

The present invention is related to analytical platforms and methods performed therewith for generating qualitative and/or quantitative protein expression profiles, in particular differential protein expression profiles, of cell populations comprising:
generating lysates of one or more populations of cells, the lysates comprising a plurality of proteins expressed by the respective cell populations,
providing an essentially planar solid support,
depositing at discrete sites small quantities of the cell lysates, in diluted or undiluted form directly on said solid support or on an adhesion-promoting layer applied on said solid support, thereby creating one or more one- or two-dimensional arrays of discrete measurement areas on said solid support,
applying a number of binding reagents as specific binding partners for the proteins contained in cell lysates in discrete measurement areas and to be detected and, if adequate, one or more detection reagents on said one or more arrays of measurement areas, the binding reagents and the detection reagents being applied sequentially or in a single addition-step, after binding of the detection reagents to the binding reagents, to the one or more arrays of discrete measurement areas, and
measuring and recording optical signals emanating from said one or more arrays of discrete measurement areas in a locally resolved manner,
wherein said essentially planar solid support is non-porous and an optionally applied adhesion-promoting layer has a thickness of less than 1 μm.

### GENERAL BACKGROUND

The present invention shall, in particular, help to improve or expedite the understanding of pharmaceutical effects and/or of toxicological effects of drugs on organisms or tissues or cell assemblies. The invention shall address, in particular, the investigation of cellular signaling cascades and the detection of the whole variety of proteins in their original or post-translationally modified forms resulting from the cellular development processes and, if adequate, from externally implied inductions to modify these processes.

The current invention shall provide an alternative to well-established protein expression profiling methods like Western blotting, capable of a higher possible through-put in time, i.e. in particular processing a large number of different samples or analyzing one or more samples for a large number of reagents applied thereto, and of delivering precise quantitative results.

For many fields of application, multiple biologically relevant analytes need to be determined in a complex sample, for example, in diagnostic methods for determining an individual's state of the health or in pharmaceutical research or development for determining the effects of the administration of biologically active compounds on an organism and on its complex functional mode.

Whereas known analytical separation methods have in general been optimized to separate the largest possible number of compounds contained in a given sample within the shortest possible time, according to a given physical-chemical parameter, such as the molecular weight or the ratio of the molecular charge and the mass, bioaffinity-related methods of determination are based on recognizing and binding with high selectivity the corresponding (single) analyte of interest in a sample of complex content by a biological or biochemical or synthetic recognition element the greatest possible specificity. The determination of many different compounds thus requires the application of a correspondingly large number of different specific recognition elements.

A determination method based on a bioaffinity reaction can be performed both in a homogeneous solution and at the surface of a solid support. Depending on the specific method, washing steps may be required after binding of the analytes to the recognition elements and of optional further tracer compounds and optionally between different steps of the process in order to separate the complexes formed between the recognition elements and the analytes to be determined and optional further tracer compounds from the residual part of the sample and of the additional indicator reagents that are optionally applied.

Methods for the simultaneous determination of many different nucleic acids in a sample using corresponding complementary nucleic acids as recognition elements immobilized in discrete, laterally separated measurement areas on a solid support are in relatively wide use nowadays. For example, arrays of oligonucleotides based on simple glass or microscope plates are known as recognition elements with a very high feature density (density of measurement areas on a common solid support). For example, in U.S. Pat. No. 5,445,934 (Affymax Technologies) arrays of oligonucleotides with a density of more than 1000 features per square centimeter have been described and claimed. Methods of this type have also found application for determining expression profiles of nucleic acids, the samples, however, typically being purified by labor-intensive methods and the modified nucleic acid samples obtained in most cases being amplified, i.e. the number of analyte molecules to be detected being biochemically enriched (multiplied) by methods like polymerase chain reaction (PCR).

Recently, there have also been frequent descriptions of similar arrays and methods based thereon for simultaneous determination of multiple proteins, for example in U.S. Pat. No. 6,365,418 B1.

The disclosures for such so-called “microarrays” for the determination both of nucleic acids and of other biopolymers, such as proteins, describe how multiple specific recognition elements are immobilized in discrete measurement areas in order to generate an array for analyte recognition and are then brought into contact with the sample to be analyzed, comprising the analytes, perhaps in a complex mixture. Following the known disclosures, different specific recognition elements are provided in as pure a form as possible in separate discrete measurement areas, so that generally different analytes will bind to measurement areas with different recognition elements.

For this kind of known assay, it is required that the specific recognition elements to be immobilized in as pure a quality as possible be enriched by means of what in some cases are very laborious steps. As different recognition elements also differ more or less in terms of their physical-chemical properties (for example, their polarity), there are also corresponding differences in the conditions for their optimized immobilization in discrete measurement areas on a common support, optionally mediated by an adhesion-promoting layer, for example, by adsorption or by covalent binding. Accordingly, the conditions chosen for immobilizing multiple different recognition elements (such as the nature of the adhesion-promoting layer) can hardly be optimal for all recognition elements to be immobilized, but will generally be a compromise between the immobilization properties of the different recognition elements of interest.

Furthermore, a disadvantage of this kind of assay is that, for the determination of analytes in a certain number of samples, it is necessary to provide a corresponding number of discrete arrays on a common support or on discrete supports to which the different samples are applied. For the analysis of multiple different samples, this implies the need for a large number of discrete arrays, the manufacture of which is relatively complex.

It has been described, for example, that under suitable conditions for dissociation the hybrids formed between immobilized oligonucleotides and complementary oligonucleotides supplied in a sample may be dissociated with high efficiency and a recognition surface thus be “regenerated”; however, a 100% regeneration can hardly be guaranteed. In the case of bioaffinity complexes with proteins, the complexation step is often not even reversible, i.e. the recognition surface cannot be regenerated.

There is therefore a need for a modified assay architecture enabling multiple samples in a single array on a common support to be analyzed for the analytes contained in said samples simultaneously. For this purpose it would be useful to immobilize not the different specific recognition elements, but the samples to be analyzed themselves, if possible directly, without further pre-treatment, or after as low a number of pre-treatment steps as possible, on a support. In the following, an assay architecture of this type shall be called an “inverted assay architecture”.

In U.S. Pat. No. 6,316,267 a method is described, wherein polyamino acids (possibly in a complex sample mixture) are, for example, applied on solid or a “semi-solid” sample matrix. The detection step, however, is performed not in a bioaffinity assay, but by staining using a mixture of reagents comprising certain metal complexes exemplified in said disclosure. This is obviously not a method of specific analyte detection.

In U.S. Pat. No. 6,287,768 a method is described, wherein different RNA molecules to be determined from a biological sample are isolated, separated by size, deposited on a solid support and then determined thereon, for example in a hybridization assay upon hybridization with known, complementary polynucleotides. According to the disclosure in that patent, either the RNA molecules to be determined and isolated from an organism can be subjected directly to the further determination method, if they are present in high abundance, or they have to be amplified beforehand by known amplification methods (e.g. by polymerase chain reaction, “PCR”).

Although the method proposed in U.S. Pat. No. 6,287,768 opens the opportunity to determine RNA from different samples simultaneously, it still requires numerous elaborate sample preparation steps and in particular isolation from the biological sample matrix, followed by a separation of the sample according to molecular size. In view of the fact that the claimed method, which is only described with reference to the example of RNA, requires at least isolation from the original sample matrix and separation of the biopolymers according to size, it has to be expected that the relative molecular composition, after this separation step and before the analysis step, will be different from the relative molecular composition of the original sample.

Here and in the following the attribute of “the same relative molecular composition” shall mean that the ratio of the concentrations of the analytes or of their modified forms (like phosphorylated, glycosylated, methylated, or acetylated forms, etc.), in the case of the present invention of proteins expressed by a cell population, to be determined in an analysis remains unchanged. Following this nomenclature, changes in the content of solvent or matrix molecules or of other molecules which are not determined in the corresponding determination method will be disregarded when using this attribute.

The fact that the detection steps applied in these methods are generally not sensitive enough for achieving required detection limits for the analytes to be determined in the samples can be seen as a reason for including the described separation or enrichment steps in the named analysis methods.

Especially in case of assays of protein immunoassays, methods are known where assays are performed using arrays of measurement areas generated on carriers having a three-dimensional surface, such as porous carriers like nitrocellulose membranes, which may be self-supporting or coated on solid supports for ease of handling, for the immobilization of the binding partners interacting with each other in the immunoassay, in order to increase the interaction surface and thus the detection sensitivity. A significant disadvantage of these three-dimensional immobilization surfaces, however, is the unavoidable delay of fluid exchange or fluid displacement from an adjacent fluid medium, thus strongly reducing the speed of the binding kinetics and strongly interfering with the removal of nonspecifically bound or adsorbed binding or detection reagents applied for analyte (i.e. relating to the scope of the invention: proteins of interest) detection, associated with the high risk of increased “background signals”, in this case mainly caused by non-specific binding or adsorption events. Additionally, for the detection of a plurality of different analytes in a common type of measurement area (i.e. of so-called deposited “spots” of the same relative molecular composition) a three-dimensional geometry of the immobilization surface bares the high risk of falsifying the detection conditions for different analytes, i.e. proteins in particular, because of spatially different distribution of the immobilized specific binding partners and spatially different conditions for the access of the applied corresponding binding and detection reagents.

Therefore, there is a need for an analytical platform enabling the generation of protein expression profiles from samples subjected to minimized sample pretreatment, in order to save costs for labor-intensive preparation steps and of required reagent volumes. Additionally, an analytical platform and a method is desired which allows to avoid the described drawbacks of three-dimensional surfaces as biochemical interaction and recognition surfaces.

A solution of these tasks is presented by the present invention.

### SHORT DESCRIPTION OF THE FIGURES

FIG. 1 shows an analytical platform according to the invention and an arrangement of 6 identical arrays of measurement areas (according to the deposited samples) on a common solid support, as an analytical platform according to the invention. The geometry of the arrangement of generated measurement areas is shown in two enlargements (see description for more explanations).

FIG. 2 shows in the left part an image of the isotropically emitted fluorescence from an array of measurement areas after incubation with 1:500 diluted Cy3-anti-β-actin (applied concentration: 6 nM; exposure time 5 sec, display range 0-20 000). Right side: Layout of array of measurement areas (1=“Non-disclosed cell laysate I” as a first control cell lysate, 2=“Non-disclosed treated cell lysate II” as a quality control for the assay performance, 3=Spotting buffer, 4=Non-treated colon cancer tissue lysate=“Tumor lysate 1”, 5=Treated colon cancer tissue lysate=“Tumor lysate 2”, 6=Empty; sample dilution increasing from left to right for each lysate, see also FIG. 1).

FIG. 3 shows a dilution plot for both non-treated and treated colon cancer tissue lysates (i.e., “Tumor lysates 1 and 2”). Data points indicate the mean net fluorescence signals of 2 replicate spots per protein concentration. Fluorescence signals were generated after incubation with a Cy3-anti-β-actin antibody (RFI=referenced fluorescence intensity).

FIG. 4 shows referenced fluorescence intensities (RFI) for the detection of β-actin in all spotted cell and tumor tissue lysates.

FIG. 5 shows bar plot profiles for protein expression measured on different arrays of measurement areas, for two colon cancer tissue lysates and two cell lysates as an internal control (each non-treated and treated), which were incubated with antibodies specific to the different signaling marker proteins (pathway activation) as specific binding reagents and then with the corresponding fluorescently labeled anti-species antibodies as detection reagents (example for path way activation).

FIG. 6 shows bar plot profiles for protein expression measured on different arrays of measurement areas, for two colon cancer tissue lysates and two cell lysates as an internal control (each non-treated and treated), which were incubated with antibodies specific to the different cell signaling marker proteins (cell proliferation).

FIG. 7 shows bar plot profiles for protein expression measured on different arrays of measurement areas, for two colon cancer tissue lysates and two cell lysates as an internal control (each non-treated and treated), which were incubated with antibodies specific to the different apoptosis marker proteins (apoptosis).

FIG. 8 shows “Fold signals” (ratio of fluorescence signals, referenced and normalized as described in section 3.4. of the example, between the signals obtained from the treated and the untreated cell populations (samples)), displayed for each protein analyte. Filled bars: fold signals, calculated from expression signals (RFI)>LOD; empty bars: fold signals, calculated from expression signals (RFI)

### DESCRIPTION OF THE INVENTION

A first subject of the present invention is a method for generating qualitative and/or quantitative protein expression profiles of one or more populations of cells comprising:
generating lysates of one or more populations of cells, the lysates comprising a plurality of proteins expressed by the respective cell populations,
providing an essentially planar solid support,
depositing at discrete sites small quantities of the cell lysates as deposited samples, in diluted or undiluted form directly on said solid support or on an adhesion-promoting layer applied on said solid support, thereby creating one or more one- or two-dimensional arrays of discrete measurement areas on said solid support,
applying a number (i.e. one or more) of binding reagents as specific binding partners for the proteins contained in cell lysates in discrete measurement areas and to be detected and, if adequate, one or more detection reagents on said one or more arrays of measurement areas, the binding reagents and the detection reagents being applied sequentially or in a single addition-step, after binding of the detection reagents to the binding reagents, to the one or more arrays of discrete measurement areas, and
measuring and recording optical signals emanating from said one or more arrays of discrete measurement areas in a locally resolved manner,

wherein said essentially planar solid support is non-porous and an optionally applied adhesion-promoting layer has a thickness of less than 1 μm.

Terms like “a” or “one” binding reagents to be applied shall always include the meaning of the application of a plurality of such compounds of the same kind in “a” or “one” applied reagent solution, if not explicitly stated otherwise.

The term “generation of protein expression profiles” shall include the determination of the absolute and/or relative number of copies of the same molecular entity for the proteins to be detected, as well as detection of such “proteins” in all forms of post-translational modifications (such as phosphorylation, glycosylation, methylation, acetylation, etc.).

Dependent on the specific purpose of the application, these different forms may be distinguished in the binding and detection steps or not distinguished (see below).

The term “qualitative” protein expression profiling shall mean determination if the corresponding proteins are present in an investigated sample (deposited cell lysate in diluted or undiluted form) or not present in such sample.

The term “quantitative” protein expression profiling shall mean that an absolute and/or relative amount of proteins of interest contained in a deposited sample is determined. Thereby, “relative” amount shall mean the amount in comparison to a reference or to a calibration sample

Subject of the invention is, in particular, a method for generating qualitative and/or quantitative differential protein expression profiles of two or more populations of cells comprising:
generating a first lysate of a population of cells, the lysate comprising a plurality of proteins expressed by the respective cell population,
generating second or more lysates of further populations of cells, the lysates comprising pluralities of proteins expressed by the respective cell population,
providing an essentially planar solid support,
depositing at discrete sites small quantities of the cell lysates as deposited samples, in diluted or undiluted form directly on said solid support or on an adhesion-promoting layer applied on said solid support, thereby creating one or more one- or two-dimensional arrays of discrete measurement areas on said solid support,
applying a number of binding reagents as specific binding partners for the proteins contained in cell lysates in discrete measurement areas and to be detected and, if adequate, one or more detection reagents on said one or more arrays of measurement areas, the binding reagents and the detection reagents being applied sequentially or in a single addition-step, after binding of the detection reagents to the binding reagents, to the one or more arrays of discrete measurement areas, and
measuring and recording a first group of optical signals emanating from the measurement areas created by deposition of small quantities of the first lysate, in diluted or undiluted form, in a locally resolved manner,
measuring and recording second or more groups of optical signals emanating from the measurement areas created by deposition of small quantities of the second or more lysates, in diluted or undiluted form, in a locally resolved manner,
comparing the measured values of the first group of optical signals with the values of the second or more groups of optical signals,

wherein said essentially planar solid support is non-porous and an optionally applied adhesion-promoting layer has a thickness of less than 1 μm.

The term “a first lysate of a population of cells” shall comprise “a plurality of first lysates of identical or different populations of cells”, the protein content of which shall be compared.

Said “first lysates may, for example, be obtained from non-treated cell populations and be used as a control sample. Said “second or more lysates of further cell populations may, for example, be obtained from cell populations that have been treated with a bioactive compound.

Correspondingly, the term “second or more lysates of further populations of cells shall comprise “pluralities of second or more lysates of further populations of cells.

Accordingly, in case of applied pluralities of applied first and/or second or more lysates the values of the corresponding multiple groups of optical signals are compared.

In the spirit of the present invention, spatially separated or discrete measurement areas on a solid support shall be defined by the closed area that is occupied by deposited lysates or deposited referencing reagents (like fluorescently labeled bovine serum albumin). These areas may have any geometry, for example the form of circles, rectangles, triangles, ellipses etc.

The term “lysate” shall be used for a liquid sample obtained from an assembly of cells derived from a cell population, provided for deposition on an analytical platform according to the invention in liquid solution. The lysates are preferably prepared in such a way that they contain the whole proteome of the cell population, cultures of cell tissues, from which they have been derived. The lysates to be deposited may be diluted in an adequate buffer solution or undiluted. It is preferred that the lysates, which are deposited at discrete sites on the solid support or on an adhesion-promoting layer on said solid support have the same relative molecular compositions of the proteins to be detected therein as the cell populations from which the lysates have been generated. Dependent on the specific application, the lysates may be further treated in a different manner, before their deposition on the solid support. The lysates may contain known additives, for example stabilizers such as enzyme inhibitors, in order to prevent a digestion of the biopolymers or their modified forms contained therein. The lysates may also contain known concentrations of compounds (as standards) similar to the analytes to be determined as additives, comparable with “spiking” of samples in chromatography. Such additives may, for example, be used for calibration purposes. Furtheron the lysates may contain additives of compounds similar to the sample matrix, such as bovine serum albumin (BSA), but different from the proteins to be detected, which may, for example be used for establishing a controlled surface density of immobilized protein molecules in a measurement area. If necessary, indissoluble material may be separated from the lysates, for example by centrifugation. It is preferred that the lysates are subjected to no further sample treatment steps than filtration and/or fractionation and/or dilution.

Terms like “a protein to be detected” or “an analyte to be detected” shall always comprise the meaning of detection of a plurality of protein or analyte molecules of the same kind, if not stated otherwise.

The proteins contained in the deposited lysates, including all forms of post-translational modifications (such as phosphorylation, glycosylation, methylation, acetylation, etc.), may be present in native or in denatured form, for example after treatment of the lysate with urea or surfactant (e.g. SDS). The proteins contained in the deposited lysates are preferably present in denatured form, after treatment with urea, such that their epitopes are freely accessible for the binding to their corresponding specific binding reagents. This is made possible by the destruction of the tertiary and quaternary structure due to the treatment with urea.

Surprisingly, the sensitivity of the method according to the invention is such that a lysate as a sample to be analyzed may even be highly diluted, and proteins contained in the mixture, in spite of their very low concentration in some cases and correspondingly small available amount in a single measurement area, can still be determined with high precision (quantitatively), which is not possible with the known conventional methods. This has the significant advantage that, in case of the method according to the invention, the deposited proteins as analytes which are contained in the sample and are to be determined are generally still present in the same relative molecular composition as in the original sample even after their immobilization. The method according to the invention can thus provide analysis results which are representative of the overall molecular composition of the original sample, because the otherwise typical enrichment and separation steps can be avoided.

In the spirit of this invention, a molecular species or compound, in particular a protein, which can be distinguished from different compounds contained in a sample to be analyzed and can be bound by a specific detection reagent applied for this purpose shall be called an “analyte”. If, for example, only the phosphorylated, but not the nonphosphorylated form of a protein shall be detected, these two forms of a protein correspond to two different analytes according to this definition. If any phosphorylated compounds or species are recognized and bound by another binding reagent, then, under these conditions, the corresponding phosphorylated compounds or species together are one analyte. According to this definition, specific binding reagents for an analyte may be selected, for example, in such a way that they exclusively recognize and bind to the phosphorylated or the glycosylated or the methylated or the acetylated (or correspondingly to the nonphosphorylated and/or nonglycosylated and/or nonmethylated and/or non-acetylated) form of a compound to be detected. The activity of a biological signal pathway in a cell or organism may be correlated with the fraction of phosphorylated or glycosylated or methylated or acetylated compounds (depending on the nature of the signal pathway) which control the corresponding signal pathway. The relative fraction of the phosphorylated and the glycosylated form, respectively, within the whole amount of the corresponding compound, i.e., the ratio of the amount of a compound present in its phosphorylated and its glycosylated form, respectively, and of the whole amount of this compound present in phosphorylated and nonphosphorylated form or in glycosylated and not glycosylated form, respectively, shall be called in the following the degree of phosporylation and the degree of glycosylation, respectively, of the corresponding compound in the sample. Similarly, the degree of methylation or acetylation shall be defined. The degree of phosphorylation and the degree of glycosylation, as well as the degree of methylation and the degree of acetylation shall be summarized under the generic term of the “degree of activation” of a compound. However, the degree of activation of a compound may also mean other, chemically modified forms of a compound.

Specific binding reagents may also be selected in such a way that they only bind to a compound (protein) to be detected if this compound (protein) is present in a certain three-dimensional structure. For example, many antibodies only recognize and bind to specific partial regions (epitopes) of a compound to be determined when they are provided in a special three-dimensional structure. Depending on the conformational state of the compound to be determined, these partial regions (epitopes) may be accessible for the binding of the corresponding binding reagents or may be hidden. The specific binding reagents may also be selected in such a way that they bind to regions of the compound to be detected, the accessibility of these regions being independent of the three-dimensional structure of the corresponding compound. Through the use of appropriately selected binding reagents it is thus possible to determine the relative amount of the total quantity of a compound which is to be detected in a sample and which shows a specific conformational state.

The method according to the invention enables a variety of different strategies for generating expression profiles of cell populations.

In one preferred embodiment, different binding reagents as specific binding partners for different proteins are applied on different arrays for each different protein to be detected. This embodiment may be performed upon additionally applying detection reagents to be attached to the binding reagents where these have bound to the proteins to be detected. This embodiment, however, may also be performed without use of such detection reagents, when, for example, the increase of surface-bound molecular mass in the measurement areas and resulting local increase of refractive index is used as a measurement method for analyte detection (see below).

In another preferred embodiment, different proteins are detected in a common array by applying different distinguishable detection reagents on said array, the number of different proteins to be detected corresponding to the number of different distinguishable labels applied. This embodiment definitively requires the application of detection reagents. In this case, the different binding reagents and detection reagents may be applied simultaneously or sequentially.

In a combination of these two embodiments, a plurality of different proteins is detected in multiple arrays of measurement areas by applying different binding reagents as specific binding partners for different proteins on different arrays for the detection of different proteins and/or different distinguishable detection reagents on the arrays of measurement areas.

Dependent on the actual scope of a study of cellular protein expression based on the present invention, the lysates to be deposited in discrete measurement areas may be selected in different manners. Characteristic for one possible embodiment of the method according to the invention, the lysates may be generated from unrelated cell populations.

“Unrelated cell population” shall denote cell populations that have not been subjected as a unity to a common cultivation process, i.e. typically cell populations originating from different organisms, organs, or cell cultures etc. grown or cultivated independent from each other. Consequently, this term shall, for example, include cell populations originating from different humans, animals or plants or organisms in general, from different organs, from different locations within such kind of organisms or organs, like cancerous and healthy tissue from the same organ, in vitro cell cultures that have been cultivated independently etc. The term shall also include, for example, cell populations that have been obtained from the same organism or organ at different points in time and/or then subjected to different treatments or types of exposure in an in vitro cultivation process. A differential expression profile generated from lysates of these unrelated cell populations may then, for example, be dedicated to monitor the differences in cell expression between different organisms, between healthy and diseased organisms of the same type, between different organisms etc., especially upon exposure to treatment with different chemical or biochemical compounds like drugs or to different growth conditions.

Characteristic for another embodiment of the method according to the invention is, that different cell lysates are generated from different cell sub-populations that have been obtained from a common cell population. For example, different cell sub-populations may have been obtained from a common cell population at different points in time. Different cell sub-populations may also have been obtained from a common cell population and then treated or stimulated with different reagents and/or exposed to different cultivation conditions. Treatment or stimulation with different reagents may include application of different chemicals or drugs to the cultures of cell sub-populations, and exposure to different cultivation conditions may include, for example, exposure to UV light, heat shock etc. as cultivation conditions applied in a generally manner not applied specifically for certain compounds contained in the exposed cell culture. In another important embodiment of the method according to the invention, different cell lysates have been generated from diseased and healthy cell populations.

The healthy or diseased and/or treated or untreated and/or stimulated cell populations from which the lysates have been generated, may have been derived from the group comprising prokaryotic cells, such as bacteria, and eukaryotic cells, such as human, animal, or plant cells, in particular human or animal tissue, such as organ, skin, hair or bone tissue, or plant tissue, and comprising cell-containing body fluids or their constituents, such as blood, serum or plasm, synovial liquids, lacrimal fluid, urine, saliva, tissue fluid, lymph.

The cell populations or parts of them used for generation of the cell lysates may have been obtained by known methods of the group including tissue slicing or biopsy, in particular micro preparation methods like laser capture micro dissection.

The samples may be deposited laterally selectively in discrete measurement areas, directly on the solid support or on an adhesion-promoting layer deposited thereon, by means of a method selected from the group of methods comprising ink jet spotting, mechanical spotting by pen, pin or capillary, “micro contact printing”, fluidic contacting of the measurement areas with the samples-through their supply in parallel or crossed micro channels, with application of pressure differences or electrical or electromagnetic potentials, and photochemical or photolithographic immobilization methods.

Following the method according to the invention, in general, several different proteins will be immobilized simultaneously in one measurement area. Typically, there will be multiple, i.e. several hundred or even several thousand, different proteins as analytes immobilized in one measurement area.

Because of the high sensitivity of the method according to the invention, it is possible to analyze even very small volumes and quantities of sample used. The quantity of sample here shall be taken to mean the total quantity of protein content which is deposited in a discrete measurement area. A sample may, for example, comprise the protein content of less than 20000 cells and still be analyzed with high precision. A sample to be deposited may even comprise the protein content of less than 1000 cells. The required sample amount may even comprise the protein content of less than 100 cells, or even the material of only 1-10 cells, and still be analyzed reliably. The protein content corresponding to the content of a single cell shall be also called a cell-equivalent. An amount of cell-equivalents such small necessary for an analysis is required when the proteins to be detected are contents of relatively high abundance. It is also possible that a sample has a volume of less than 1 μl. A sample to be may even have a volume of less than 10 nl or even less than 1 nl.

Especially to facilitate the analysis procedure to determine and/or compare the protein expression profiles of cell populations when different binding reagents as specific binding partners for different proteins to be detected are applied on different arrays for each protein to be detected, if adequate combined with the application of one or more detection reagents of which, if distinguishable, in number of two or more may be applied to the same array, it is advantageous, if replicates of the same array of measurement areas are provided on a common solid support: In a farther preferred embodiment of the method according to the invention are therefore multiple arrays of measurement areas arranged in an identical geometry of the deposition sites of the diluted or undiluted cell lysates, a similar position with respect to rows and column of a measurement area in two different arrays corresponding to deposited amounts from the same (diluted or undiluted) cell lysate deposited therein.

In general, the simplest method for immobilizing the specific binding partners for an analyte determination in a specific binding reaction is physical adsorption, for example, based on hydrophobic interactions between the specific binding partners to be immobilized and the solid support. The strength of these interactions, however, may be markedly changed by the composition of the medium and its physical/chemical properties, such as polarity and ionic strength. Especially in the case of the sequential supply of different reagents in a multi-step assay, the adhesion of the recognition elements, in case of the present invention proteins contained in the deposited lysates, is often insufficient after purely adsorptive immobilization on the surface. It is therefore preferred if the solid support comprises an adhesion-promoting layer on which the samples are deposited, in order to improve their adhesion.

The adhesion-promoting layer has a thickness of preferably less than 200 nm, especially preferably less than 20 nm.

Various materials are suitable for generating the adhesion-promoting layer. For example, the adhesion-promoting layer may comprise compounds of the group of silanes, functionalized silanes, epoxides, functionalized, charged or polar polymers and “self-organized passive or functionalized mono- or multi-layers”, thiols, alkyl phosphates and alkyl phosphonates, multi-functional block copolymers, such as poly(L)lysin/polyethylene glycols.

Said adhesion-promoting layer may also comprise compounds of the group of organophosphoric acids of the general formula I(A)
Y—B—OPO₃H₂ (IA)

or of organophosphonic acids of the general formula I(B)
Y—B—PO₃H₂ (IB)

and of their salts, wherein B is an alkyl, alkenyl, alkinyl, aryl, aralkyl, hetaryl, or hetarylalkyl residue, Y is hydrogen or a functional group of the following series, e.g. hydroxy, carboxy, amino, mono- or dialkyl amino optionally substituted by lower alkyl, thiol, or negative acidic group of the following series, e.g. ester, phosphate, phosphonate, sulfate, sulfonates, maleimide, succinimydyl, epoxy or acrylate. These compounds have been described in more detail in the international patent application PCT/EP 01/10077, which is hereby incorporated in this disclosure in its whole entirety.

The method according to the invention is preferably designed in such a way that the relative molecular composition of the samples (diluted or indiluted cell lysates) immobilized in a measurement area is identical with the original relative molecular composition of the sample applied to said measurement area. This requirement may, for example, be met if the material amount of a sample deposited in a measurement area is equal to or less than the amount of material necessary for the formation of a monolayer on the solid support. At the same time, an optimum accessibility of the proteins as analytes for the binding reagents and, if adequate, additional detection reagents to be brought into contact with them is given in case of a sub-monolayer coverage of the surface of the solid support. The accessibility may be even further improved if the adhesion-promoting layer deposited beforehand leads to an oriented immobilization, for example if antibodies contained in the deposited sample are immobilized bound to their F_{C}-part, resulting in accessibility of their specific binding epitopes.

The method according to the invention allows to determine the relative total amounts of one or more compounds contained as analytes in a deposited sample, as the sum of their occurrence in phosphorylated or not phosphorylated form and/or glycolisated and/or not glycolisated form. It is preferable if the relative amounts of one or more compounds contained as analytes in a deposited sample, in each case of their occurrence in phosphorylated and/or nonphosphorylated form and/or glycosylated and/or nonglycosylated form, are determined for one or more said forms.

The method according to the invention allows the degree of activation, as defined above, of one or more analytes contained in a sample to be determined. In particular, the method according to the invention allows the degree of phosphorylation and/or the degree of glycosylation and/or the degree of methylation and/or the degree of acetylation of one or more analytes contained in a sample to be determined. As a result of the high sensitivity and high precision and reproducibility, in particular as a result of the numerous independent referencing and calibration methods that can be applied simultaneously or alternatively, it is also characteristic of the method according to the invention that differences of less than 20%, preferably less than 10%, between the relative amounts of one or more compounds contained in phosphorylated and/or nonphosphorylated and/or glycosylated and/or nonglycosylated and/or methylated and/or non-methylated and/or acetylated and/or non-acetylated form as analytes in a first sample and in one or more comparison samples can be determined for one or more of said forms. The method according to the invention also allows for the determination of small differences, e.g. of 40%, preferably of less than 30%, most preferably of less than 10% in respective protein concentrations, in the protein expression profiles by measurement of the lysates from different related or unrelated cell populations.

As a result of the inherent, method-specific high sensitivity and the diversity of possibilities for referencing and/or calibration using one and the same analytical platform, it is an important advantage of the method according to the invention that the variation of the measurement results obtained with this method is very low. The method according to the invention is thus also suitable for investigating the temporal evolution (i.e. the changes) of the relative amounts or concentrations of contained proteins influenced by a disease of a biological organism or of a cell culture and/or upon external manipulation of an organism or a cell culture.

It is of advantage if regions between the discrete measurement areas are “passivated” in order to minimize nonspecific binding of tracer compounds, i.e. that compounds which are “chemically neutral” (i.e. nonbinding) towards the analytes (i.e. proteins) and the other contents of the deposited samples and the binding and/or detection reagents for said analytes (i.e. proteins) are deposited between the laterally separated measurement areas.

Said compounds which are “chemically neutral” (i.e. nonbinding) towards the analytes (i.e. proteins) and the other contents of the deposited samples and the binding and/or detection reagants for said analytes may be selected from the group comprising albumins, especially bovine serum albumin or human serum albumin, casein, nonspecific, polyclonal or monoclonal, heterologous or empirically nonspecific antibodies (for the analytes to be determined, especially for immunoassays), detergents—such as Tween 20—, fragmented natural or synthetic DNA not hybridizing with polynucleotides to be analyzed, such as extracts of herring or salmon sperm, or also uncharged but hydrophilic polymers, such as polyethyleneglycols or dextrans.

Without loss of generality, the proteins as analytes which are to be detected and are contained in the samples deposited in discrete measurement areas may be compounds of the group of proteins comprising cytosolic, nuclear and membrane proteins, secreted proteins in body fluids (cytosolic and membrane-bound cell proteins, especially proteins involved in the processes of signal transduction in cells, such as kinases), post-translationally modified proteins like phosphorylated, glycosylated, methylated, and acetylated forms of proteins, in particular proteins over- and or under-expressed under treatment, said group comprising antibodies, artificially overexpressed proteins, artificially overexpressed modified proteins like functionalized proteins with additional binding sites (“tag proteins”, such as “histidine tag proteins”), and fluorescent proteins (“green fluorescent proteins”, GFP and the like). The analytes may be biotechnologically modified polymers, e.g. biologically expressed biopolymers comprising luminescent or fluorescent groups, respectively, such as “blue fluorescent proteins” (BFP), “green fluorescent proteins” (GFP), or “red fluorescent proteins” (RFP).

According to one embodiment of the method according to the invention, the proteins which are to be detected and are contained in the diluted or undiluted lysates deposited in discrete measurement areas are distinguished in the step of binding added specific binding reagents and, if adequate, detection reagents, added sequentially or in a single addition step, after binding of the detection reagents to the binding reagents, according to their occurrence in phosphorylated and/or nonphosphorylated form and/or glycosylated and/or nonglycosylated form contained in the diluted or undiluted deposited lysates to be analyzed.

In another embodiment, the proteins which are to be detected and are contained in the diluted or undiluted lysates deposited in discrete measurement areas are not distinguished in the step of binding added specific binding reagents and, if adequate, detection reagents, added sequentially or in a single addition step, after binding of the detection reagents to the binding reagents, between their occurrence in phosphorylated or nonphosphorylated form and/or glycosylated or nonglycosylated form contained in the diluted or undiluted deposited lysates to be analyzed.

The specific binding reagents for the analytes to be detected in the discrete measurement areas may be selected from the group of compounds binding specifically to the mentioned protein analytes, like antibodies to antigens and vice versa, anti-species antibodies for species antibodies, etc., as well-known for the expert in the art.

The detection reagents may be selected from the group of reagents or labels dedicated for, e.g., specific optical detection methods, comprising ESR spin labels for measurements based on locally resolved electron spin resonance (ESR) measurements, nuclear magnetic resonance (NMR) labels or measurements based on locally resolved nuclear magnetic resonance (NMR) measurements, radioactive labels for measurements of radioactive isotopes as labels, mass labels, like beads for locally resolved measurements of refractive index changes due to desorption or adsorption of molecular mass on the measurement areas, luminescence labels, in particular fluorescence labels (which will be further specified below). These labels may be applied subsequently after addition of the binding reagents to the arrays of measurement areas or after binding of first detection reagents (like anti-species antibodies applied as detection reagents to analyte-specific antibodies) or be an integral part of the detection reagents. The labels may also be attached directly to the binding reagents.

It is preferred if the material of the essentially planar solid support being in physical contact with the generated measurement areas either directly or mediated by an adhesion promoting layer is essentially optically transparent.

It is also preferred if the material of an adhesion layer applied on the solid support is likewise essentially optically transparent.

Preferably, the material of the essentially optically transparent solid support comprises a material from the group comprising moldable, sprayable or millable plastics, metals, metal oxides, silicates, such as glass, quartz or ceramics.

Depending on the physical design of the solid support, there are several possibilities for the metrological type of signal generation in analyte determination. In general, a method is preferred wherein probing light from one or more polychromatic or monochromatic light sources is directed towards one or more measurement areas in one or more arrays of measurement areas and optical signals emanating from said one or more arrays of measurement areas and/or changes in these optical signals are measured and recorded.

Characteristic for one group of embodiments of the method is that the probing light is delivered in an epi-illumination configuration.

Characteristic for another group of embodiments is that the probing light is delivered in a trans-illumination configuration.

Preferably, the detection of one or more proteins in discrete measurement areas is based on the detection of the intensities or changes in the intensities of one or more luminescences.

Characteristic for a special group of embodiments, according to the method of signal detection, is that the detection of one or more proteins in discrete measurement areas is based on the detection of changes in the refractive index on said measurement areas or within a distance of less than 1 μm from these measurement areas.

Within this group of special embodiments of the method according to the invention, one variant is characterized in that the detection of changes in the refractive index on said measurement areas or within a distance of less than 1 μm from these measurement areas is based on detection of changes in the pattern of interferences of light emanating from the planar solid support in the regions of the measurement areas generated on the solid support with light emanating from planes of interfaces to materials of different refractive index, caused by changes of the phase differences between the light emanating from said interfaces and the light emanating from the regions of the measurement areas due to binding or desorption or displacement of applied specific binding partners, and wherein the interference light emanating from the different regions is measured in a locally and, if adequate, spectrally resolved manner. The measurement method of this embodiment is based on the well-known principle of interference of light emanating from different parallel thin layers of different refractive index, which can be exploited by determining, in a locally resolved manner (with respect to the solid support carrying the arrays of measurement areas), the phase differences and their changes induced by the binding of binding reagents specific for the proteins to be detected and, if adequate, additionally applied detection reagents, and/or the spectral change of the interference pattern.

Characteristic for another variant within this group of embodiments of the method according to the invention is that the solid support is provided with a thin metal layer, preferably of silver or gold and preferably with a thickness between 20 nm and 200 nm, which is directly or mediated by an adhesion-promoting layer in contact with the measurement areas, and the detection of changes in the refractive index on said measurement areas or within a distance of less than 1 μm from these measurement areas is based on detection of changes in the conditions for generating a surface plasmon resonance in said metal layer.

As techniques of measurement, the resonance angle (upon variation of the incidence angle of the irradiated light at constant wavelength) and the resonance wavelength (upon variation of the irradiated excitation wavelength at constant incidence angle) can be measured for the determination of changes in the resonance conditions. Consequently, said change in the resonance conditions may be manifested by a change in the resonance angle for the irradiation of an excitation light for generation of a surface plasmon in a thin metal layer as part of said solid support. Accordingly, said change in the resonance conditions may also be manifested by a change in the resonance wavelength of an irradiated excitation light for generation of a surface plasmon in a thin metal layer as part of said solid support.

As a consequence of the binding of specific binding and/or detection reagents to proteins as analytes contained in the samples in discrete measurement areas, the changes in optical signals to be determined in laterally resolved manner may be caused by local changes in the effective refractive index in these regions on said solid supports when provided as evanescent field sensor platform.

In another preferred embodiment of the method according to the invention, the solid support comprises a continuous optical waveguide or an optical waveguide divided into individual waveguiding areas.

It is especially preferred if the optical waveguide is an optical film waveguide with a first essentially optically transparent layer (a) facing the surface carrying the discrete measurement areas on a second essentially optically transparent layer (b) with a refractive index lower than that of layer (a).

Thereby preferred is an embodiment, wherein, for the in-coupling of probing light into the optically transparent layer (a), this layer is in optical contact with one or more optical in-coupling elements from the group comprising prism couplers, evanescent couplers with combined optical waveguides with overlapping evanescent fields, butt-end couplers with focusing lenses, preferably cylinder lenses, arranged in front of one face of the waveguiding layer, and grating couplers.

Especially preferred is, if the probing light is in-coupled into the optically transparent layer (a) using one or more grating structures (c) which are featured in the optically transparent layer (a).

Characteristic for another variant of detection of (local) changes of the effective refractive index is that light guided in the optically transparent layer (a) is out-coupled using one or more grating structures (c′) which are featured in the optically transparent layer (a). In this case, the change of the out-coupling angle (caused by changes of the molecular mass on the out-coupling grating) may be used as the measurement parameter.

Both in configurations for generating a surface plasmon resonance (as described above) and of coupling an excitation light into a waveguiding layer, either the wavelength of the delivered light may be kept constant and the angle for matching the resonance conditions may be varied and recorded when maximum resonance (or in-coupling into a waveguiding layer) is achieved, or the incidence angle may be kept constant, and the irradiated wavelength may be varied (e.g. using a spectrally tunable laser or laser diode), and the wavelength when matching the resonance conditions may be measured and recorded.

It is preferred if the changes in optical signals which are to be determined laterally resolved, as a consequence of the binding of binding and optionally applied detection reagents to analytes contained in the samples in discrete measurement areas, being caused by local changes in one or more luminescences from molecules capable of luminescence, which are located on the solid support

It is strongly preferred if the optical waveguide is designed as an optical film waveguide with a first optically transparent layer (a) on a second optically transparent layer (b) with lower refractive index than layer (a), wherein probing light is further in-coupled into the optically transparent layer (a) with the aid of one or more grating structures, which are featured in the optically transparent layer (a), and delivered as a guided wave to measurement areas (d) located thereon, and wherein the luminescence of molecules capable of luminescence, generated in the evanescent field of said guided wave, is further determined using one or more detectors, and the relative amount of proteins contained in the measurement areas is determined from the intensity of these luminescence signals.

It is preferred if luminescences or the changes in one or more luminescences originate from molecules or nanoparticles capable of luminescence, which are bound as luminescence labels to one or more detection reagents or to one or more binding reagents for the proteins as analytes contained in discrete measurement areas.

It is preferred if luminescences are generated upon excitation of detection reagents associated with binding reagents that have specifically bound to proteins to be detected in the measurement areas, and wherein the detection reagents comprise luminescent dyes or luminescent nanoparticles used as luminescence labels, which can be excited and emit at wavelengths between 300 nm and 1100 nm.

It is especially advantageous if two or more luminescence labels with different emission wavelengths and/or different excitation spectra, preferably with different emission wavelengths and identical excitation wavelength, are applied for analyte detection. If several luminescence labels with different spectral properties, especially with different emission wavelengths, are bound to different detection reagents or directly to the binding reagents, which are brought into contact with the measurement areas, for example, different analytes can be determined in a single detection step, i.e. when the measurement areas are brought into contact with said detection reagents and the generated luminescences are detected simultaneously or consecutively, if necessary upon launching of probing light (or excitation light, respectively) of different wavelengths.

Such a variant of the method according to the invention is, for example, especially suitable for simultaneously detecting, for example, the phosphorylated and the nonphosphorylated form of a compound (protein), especially also within one (common) measurement area, by using two correspondingly different distinguishable detection reagents.

In a similar way, two or more analytes can be detected simultaneously if two or more luminescence labels (as total or integral part of the detection reagents) with different emission decay times (emission lifetimes) are applied for analyte detection.

For the method according to the invention, it is therefore preferred if two or more luminescence labels are applied for detecting different analytes in a sample. It is also preferred if two or more luminescence labels are applied for detecting different analytes in a measurement area.

It is also advantageous if the excitation light is irradiated in pulses with a duration between 1 fs and 10 minutes, and the emission light from the measurement areas is measured in a time-resolved manner.

A special variant consists in changes in the effective refractive index on the measurement areas being determined in addition to the determination of one or more luminescences.

For a further improvement in sensitivity it can be advantageous here if the determinations of one or more luminescences and/or determinations of light signals at the excitation wavelength are performed as polarization-selective measurements. It is preferred here if the one or more luminescences are measured at a polarization that is different from the polarization of the excitation light.

A further subject of the invention is an analytical platform for optical signal read-out and for generating qualitative and/or quantitative protein expression profiles of one or more populations of cells comprising:
an essentially planar solid support,
one or more one- or two-dimensional arrays of discrete measurement areas on said solid support, said arrays being generated by deposition of small quantities of cell lysates, in diluted or undiluted form, at discrete sites directly on said solid support or on an adhesion-promoting layer applied on the solid support before, the cell lysates originating from one or more populations of cells and containing a plurality of proteins expressed by these cell populations,

wherein said essentially planar solid support is non-porous and an optionally applied adhesion-promoting layer has a thickness of less than 1 μm.

A particular subject of the invention is an analytical platform for optical signal read-out and for generating qualitative and/or quantitative differential protein expression profiles of one or more populations of cells comprising:
an essentially planar solid support,
one or more one- or two-dimensional arrays of discrete measurement areas on said solid support, said arrays being generated by deposition of small quantities of two or more cell lysates, in diluted or undiluted form, at discrete sites directly on said solid support or on an adhesion-promoting layer applied on the solid support before, the cell lysates originating from two or more populations of cells and containing a plurality of proteins expressed by these cell populations,

wherein said essentially planar solid support is non-porous and an optionally applied adhesion-promoting layer has a thickness of less than 1 μm.

Different deposited cell lysates may have been generated from unrelated cell populations as defined above.

Different deposited lysates may also have been generated from different cell sub-populations that have been obtained from a common cell population.

Characteristic for one variant of an analytical platform according to the invention is that different deposited lysates have been generated from different cell sub-populations that have been obtained from a common cell population at different points in time.

Different deposited lysates may also have been generated from different cell sub-populations that have been obtained from a common cell population and then treated or stimulated with different reagents and/or exposed to different cultivation conditions.

In particular, different deposited lysates may have been generated from diseased and healthy cell populations.

The healthy or diseased and/or treated or untreated and/or stimulated cell populations from which the deposited lysates have been generated may have been derived from the group comprising prokaryotic cells, such as bacteria, and eukaryotic cells, such as human, animal, or plant cells, in particular human or animal tissue, such as organ, skin, hair or bone tissue, or plant tissue, and comprising cell-containing body fluids or their constituents, such as blood, serum or plasm, synovial liquids, lacrimal fluid, urine, saliva, tissue fluid, lymph.

It is preferred if the lysates, in diluted or undiluted form, that are deposited at discrete sites on the solid support or on an adhesion-promoting layer on said solid support have the same relative molecular compositions of the proteins to be detected therein as the cell populations from which the lysates have been generated.

It is especially preferred if the deposited lysates have been subjected to no further sample treatment steps than filtration and/or fractionation and/or dilution.

Due to the high detection sensitivity provided by the analytical platform according to the invention, the material deposited in a single measurement area may correspond to the protein content of less than 1000 cells.

An advantageous embodiment of an analytical platform according to the invention is one wherein an array comprises more than 50, preferably more than 500, most preferably more than 5000 measurement areas.

Each measurement area here may comprise an immobilized “nature-identical” sample or comparison sample which is similar to or different from the samples immobilized in other measurement areas.

The measurement areas of an array may be arranged in a density of more than 10, preferably more than 100, most preferably more than 1000 measurement areas per square centimeter.

Especially to facilitate the analysis procedure to determine and/or compare the protein expression profiles of cell populations when different binding reagents as specific binding partners for different proteins to be detected are applied on different arrays for each protein to be detected, if adequate combined with the application of one or more detection reagents of which, if distinguishable, in number of two or more may be applied to the same array, it is advantageous, if replicates of the same array of measurement areas are provided on a common solid support: In a farther preferred embodiment of the analytical platform according to the invention are therefore multiple arrays of measurement areas arranged in an identical geometry of the deposition sites of the diluted or undiluted cell lysates, a similar position with respect to rows and column of a measurement area in two different arrays corresponding to deposited amounts from the same (diluted or undiluted) cell lysate deposited therein.

It is preferred if an adhesion-promoting layer applied on the solid support has a thickness of less than 200 nm, preferably of less than 20 nm.

The adhesion-promoting layer may comprise compounds of the group of silanes, functionalized silanes, epoxides, functionalized, charged or polar polymers and “self-organized passive or functionalized mono- or multi-layers”, thiols, alkyl phosphates and alkyl phosphonates, multi-functional block copolymers, such as poly(L)lysin/polyethylene glycols.

Said adhesion-promoting layer may also comprise compounds of the group of organophosphoric acids of the general formula I(A)
Y—B—OPO₃H₂ (IA)

or of organophosphonic acids of the general formula I(B)
Y—B—PO₃H₂ (IB)

and of their salts, wherein B is an alkyl, alkenyl, alkinyl, aryl, aralkyl, hetaryl, or hetarylalkyl residue, Y is hydrogen or a functional group of the following series, e.g. hydroxy, carboxy, amino, mono- or dialkyl amino optionally substituted by lower alkyl, thiol, or negative acidic group of the following series, e.g. ester, phosphate, phosphonate, sulfate, sulfonates, maleimide, succinimydyl, epoxy or acrylate. These compounds have been described in more detail in the international patent application PCT/EP 01/10077, which is hereby incorporated in this disclosure in its whole entirety.

Embodiments of the analytical platform according to the invention are characterized in that regions between the discrete measurement areas are “passivated” in order to minimize nonspecific binding of tracer compounds, i.e. that compounds which are “chemically neutral” (i.e. nonbinding) towards the binding reagents and, if adequate, towards the detection reagents are deposited between the laterally separated measurement areas.

The proteins which are to be detected and are contained in the diluted or undiluted lysates deposited in discrete measurement areas may be compounds of the group of proteins comprising cytosolic, nuclear and membrane proteins, secreted proteins in body fluids (cytosolic and membrane-bound cell proteins, especially proteins involved in the processes of signal transduction in cells, such as kinases), post-translationally modified proteins like phosphorylated, glycosylated, methylated, and acetylated forms of proteins, in particular proteins over- and or under-expressed under treatment, said group comprising antibodies, artificially overexpressed proteins, artificially overexpressed modified proteins like functionalized proteins with additional binding sites (“tag proteins”, such as “histidine tag proteins”), and fluorescent proteins (“green fluorescent proteins”, GFP and the like).

It is preferred if the material of the essentially planar solid support being in physical contact with the generated measurement areas either directly or mediated by an adhesion promoting layer is essentially optically transparent.

Likewise it is preferred if the material of an adhesion layer applied on the solid support is also essentially optically transparent.

Preferably, the material of the essentially optically transparent solid support comprises a material from the group comprising moldable, sprayable or millable plastics, metals, metal oxides, silicates, such as glass, quartz or ceramics.

In another embodiment of the analytical platform according to the invention, the solid support is provided with a thin metal layer, preferably of silver or gold and preferably with a thickness between 30 nm and 200 nm, which is directly or mediated by an adhesion-promoting layer in contact with the measurement areas, the platform being operable for generating a surface plasmon resonance in said metal layer. A special variant of an analytical platform according to the invention comprises the evanescent field sensor platform, as part of the analytical platform, comprises a thin metal layer, optionally on an intermediate layer with refractive index preferably <1.5, such as silicon dioxide or magnesium fluoride, located beneath, and wherein the thickness of the metal layer and of the optional intermediate layer are selected in such a way that a surface plasmon can be excited at the wavelength of an irradiated excitation light and/or of a generated luminescence.

It is preferred here if the metal is selected from the group comprising gold and silver. It is also preferred if the metal layer has a thickness between 10 nm and 1000 nm, preferably between 30 nm and 200 nm.

In another embodiment, preferably, the solid support comprises a continuous optical waveguide or an optical waveguide divided into individual waveguiding areas.

It is preferred if the optical waveguide is an optical film waveguide with a first essentially optically transparent layer (a) facing the surface carrying the discrete measurement areas on a second essentially optically transparent layer (b) with a refractive index lower than that of layer (a).

Particularly preferred is an analytical platform according to the invention comprising an analytical platform wherein, for the in-coupling of probing light into the optically transparent layer (a), this layer is in optical contact with one or more optical in-coupling elements from the group comprising prism couplers, evanescent couplers with combined optical waveguides with overlapping evanescent fields, butt-end couplers with focusing lenses, preferably cylinder lenses, arranged in front of one face of the waveguiding layer, and grating couplers.

It may be advantageous if one or more grating structures (c) are featured in the optically transparent layer (a) for allowing in-coupling of probing light into the optically transparent layer (a).

The invention further comprises an analytical platform comprising an optical film waveguide wherein said optical waveguide is designed as an optical film waveguide with a first optically transparent layer (a) on a second optically transparent layer (b) with lower refractive index than layer (a), and wherein the analytical platform is operable of in-coupling probing light into the optically transparent layer (a) with the aid of one or more grating structures, which are featured in the optically transparent layer (a), delivering said probing light as a guided wave to measurement areas (d), and exciting luminescence of molecules capable of luminescence in the evanescent field of said guided wave.

A further subject of the invention is the use of a method according to any of the above embodiments and/or of an analytical platform according to any of the embodiments described above for quantitative and/or qualitative analyses for the determination of proteins in screening methods in pharmaceutical research, combinatorial chemistry, clinical and pre-clinical development, for real-time binding studies and the determination of kinetic parameters in affinity screening and in research, especially for the determination of proteomic differences in the proteome, for the measurement of protein-DNA interactions, for the determination of control mechanisms for the protein (bio)synthesis, for the screening of biological and chemical marker compounds, for patient stratification

In the following, the invention is further explained by examples of applications. The embodiments herein do not imply any loss of generality.

The method according to any of the above embodiments and/or of an analytical platform according to any of the embodiments described above is, in particular suited for high through-put profiling of pathway activation markers upon correlating the effect of modifications of proteins on their expression, for profiling and/or screening of compounds and/or drug candidates in drug discovery for their efficacy and/or toxicity upon application to cell cultures (populations) as models for organisms for disease-related applications, for biomarker monitoring, biomarker discovery and validation, pharmaceutical target discovery, validation and monitoring (e.g., upon correlating protein expression/expression activation with the response to applied drugs), determination of cell- or tissue-specific protein expression and/or activation of protein expression of, for example, cancer cells in different development states (e.g. cancerous pre-early and advanced stages), for the correlation of protein expression profiles and their changes with biological events, for so-called “global analysis” of signaling pathways, and for screening sets or libraries of antibodies against protein targets contained in the lysate samples for best specificity, selectivity and affinity.

### EXAMPLE

1. Materials

1.1. Tissue Lysate Samples

Two different tissue lysates were used for which differential protein expression profiles should be established. The lysates had been obtained from cell sub-populations that had been derived from a common cell population: Cancerous tissue (=common cell population) had been divided into two cell sub-populations that had then been cultivated independent from each other. One of them was subjected to no further treatment and was used to generate a control sample (“tumor lysate 1”). The other cell sub-population was treated chemically and then used to generate a second lysate sample (“tumor lysate 2”). The samples had the following characteristics:

| Tissue | Treatment | Protein concentration |
|---|---|---|
| 1. Colorectal cancer: | None (control) | 2.9 mg/ml |
| “Tumor lysate 1” | | |
| 2. Colorectal cancer: | Chemotherapy | 2.6 mg/ml |
| “Tumor lysate 2” | | |

Protein concentrations were determined according to a modified Bradford test, using a PIERCE Coomassie Plus-Kit (see section 3.1)

1.2. Antibodies and Assay Reagents

The following marker-specific antibodies were selected and used as specific binding reagents (CST=Cell Signaling Technology, Inc., Beverly, Mass. 01915, USA, BD=BD Biosciences, Basel, Switzerland):
α-P-p44/42 MAPK (CST #9101) rabbit
α-P-Akt (CST #4051) mouse IgG2b
α-P-p38 (CST #9211) rabbit
α-P-SAPK/JNK (CST #9251) rabbit
α-P-IκB-α (CST #9241) rabbit
α-P-Stat3 (CST #9138) mouse IgG1
α-P-Histone H3 (CST #9706) mouse IgG1
α-P—Rb (CST #9308) rabbit
α-P-p53/Ser15 (CST #9284) rabbit
α-Cyclin D1 (CST #2926) mouse IgG2a
α-Cleaved caspase3 (CST #9664) rabbit
α-Cleaved PARP (CST #9541) rabbit
α-α-catenin (BD #6101193)
α-β-catenin (CST #9562)

The following fluorescently labelled compound was used as a detection reagent for quality control of tissue lysate arrays:
Cy3-α-β-actin (in-house-labeled)

The following fluorescently labeled anti-species antibodies were applied as detection reagents:
Alexa Fluor 647 (α-rabbit IgG); Molecular Probes #Z-25308
Alexa Fluor 647 (α-mouse IgG1); Molecular Probes #Z-25008
Alexa Fluor 647 (α-mouse IgG2a); Molecular Probes #Z-25108
Alexa Fluor 647 (α-mouse IgG2b); Molecular Probes #Z-25208

All analyte-specific antibodies were used at a 1:250 dilution in assay buffer containing 5% bovine serum albumin (BSA) or 5% fat free milk powder, according to the suppliers recommendations. Detection reagents (fluorecence-labelled Fab fragments) were used at a 1:500 dilution in assay buffer containing 5% BSA.

2. Solid Support as Part of an Analytical Platform According to the Invention

As essentially planar solid supports as part of analytical platforms according to the invention planar film waveguides were used, which had the dimensions of 14 mm width ×57 mm length×0.7 mm thickness. These thin-film waveguides comprise a glass substrate (AF 45 as second optically transparent layer (b)) and a 150 nm thin, highly refractive layer of tantalum pentoxide (as first optically transparent layer (a) deposited thereon. Two surface relief gratings (grating structures (c) and (c′), in parallel to the length of these plates, are modulated in the glass substrate at a distance of 9 mm between each other (grating period: 318 nm, grating depth: 12 nm±2 nm). These structures, which serve as diffractive gratings for the in-coupling of light into the highly refractive layer, are carried over into the surface of the tantalum pentoxide layer in the subsequent deposition of the highly refractive layer.

After careful cleaning of the thin-film waveguide plates, a monolayer of mono dodecyl phosphate (DDP), as an adhesion-promoting layer, was generated on the surface of the metal oxide layer by spontaneous self-assembly, upon precipitation from an aqueous solution (0.5 mM DDP). This surface modification of the initially hydrophilic metal oxide surface leads to a hydrophobic surface (with a contact angle of about 100° against water), on which multiple (diluted or undiluted) lysate samples should be deposited.

3. Methods—Tissue Lysate Array Production, Assay Preparation and Data Analysis

3.1. Protein Quantification

Protein concentration of tissue lysates was determined with the PIERCE Coomassie Plus Kit (PIERCE #23238) using BSA in 10-fold diluted lysis buffer as a standard. The tissue lysates were diluted by a factor 10 in phosphate-buffered saline (PBS) prior to addition of the colorimetric reagent. Results are given in Section 1.1

3.2. Generation of Measurement Areas and Array Geometry

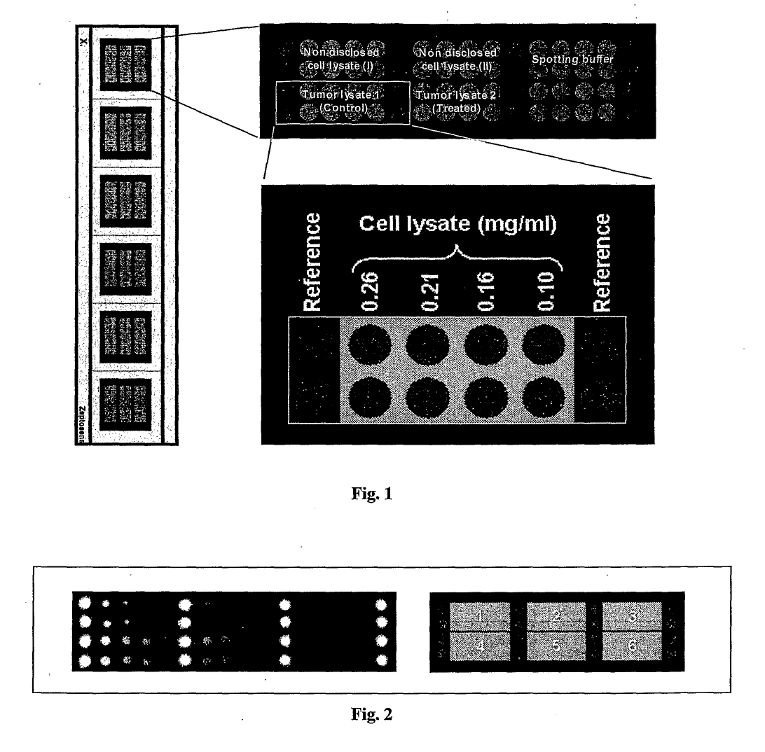

Tissue lysates were diluted to final lysate concentrations for deposition (“spotting”) on the solid support provided with an adhesion-promoting layer as described in section 2. The protein concentrations of the spotting solutions were 0.26 mg/ml, 0.21 mg/ml, 0.16 mg/ml, and 0.10 mg/ml, respectively, in urea containing spotting buffer. Discrete measurement areas (“spots”) were generated on the solid support by deposition of single droplets of about 400 pl volume using an ink jet spotter. Two replicate spots from each lysate solution were generated adjacent to each other in a common column, and within one row, always four measurement areas were generated from the four different lysate dilutions (see FIG. 1). Two additional, differently treated cell lysates (denoted as “non-disclosed cell lysates” in the upper enlarged view of FIG. 1) were spotted as an internal positive control for the assay performance. The positive controls typically reveal up-regulation of the signaling pathway marker proteins P-Akt and P-Erk2 levels upon treatment. Measurement areas dedicated for a negative control were generated by spotting buffer solution (not containing any cell or tissue lysates).

In addition to the measurement areas comprising deposited lysate samples and buffer solution, respectively, each array comprised additional measurement areas containing immobilized bovine serum albumin fluorescently labeled with Cy5 (Cy5-BSA), which were used for referencing local differences and/or temporal variations of the excitation light intensity (denoted as “Reference” in the lower enlarged view in FIG. 1). Cy5-BSA (labeling rate: about 3 Cy5 molecules per BSA molecule) was deposited at a concentration of 0.5 nM in 3.5M Urea, 1M Thiourea. Fluorescence signals from these reference spots were used for signal normalization in order to compensate for differences of the excitation light intensity within and between arrays.

After generation of the tissue lysate arrays, the free hydrophobic regions on the platform not coated with protein were saturated with bovine serum albumin (BSA) by incubation of the surface with a solution of BSA (30 mg/ml) in 50 mM imidazole/100 mM NaCl (pH 7.4), BSA being used as a compound that is “chemically neutral” (i.e. nonbinding) towards the analytes and the other contents of the deposited samples and the binding and detection reagents to be applied, in order to minimize nonspecific binding to the surface. The analytical platform was then washed with purified water, dried in a stream of nitrogen and stored in the dark at 4° C. until use.

Each analytical platform comprised six identical arrays of measurement areas. A fluidic structure was attached to the surface of the analytical platform, as described in the international patent applications WO 01/43875 and WO 02/103331, in order to generate an arrangement of six sample compartments with an inner volume of 15 μl, each containing one of the six arrays of measurement areas.

Assay Procedure

A quality control of the lysate arrays, i.e. a determination of missing spots, spot shape and homogeneity as well as applied relative protein concentration was performed by detection of β-actin as a housekeeping protein. The detection of β-actin was performed in a single step assay by addition of 500-fold diluted Cy3-α-β-actin antibody in assay buffer containing 5% BSA onto one array of measurement areas (applied concentration: 6 nM), followed by an incubation for 1 hour at 25° C. After removal of excess fluorescently labeled antibody with assay buffer containing 5% BSA, the array was subjected to the detection step by means of excitation and detection of the resulting fluorescence signals using the ZeptoREADER™ (see below).

The detection of analytes (marker-specific proteins) in the tissue lysate spots was performed in a two step sequential assay. The first step comprised the addition of analyte-specific antibodies as specific binding reagents (see section 1.2) to the arrays of measurement areas and incubation over night (at 25° C.). In this case, always only one binding reagent was applied to each individual array, so that application of the listed 14 different binding reagents required the application to 14 different arrays. After removal of excess antibody, the arrays were incubated with fluorescently labeled anti-species Fab-fragments as detection reagents for 1 hour at 25° C. A common fluorescence label (Alexa Fluor 647) was used for signal generation, that was attached to the different anti-species antibodies. According to their specificity of species, different anti-species antibodies were applied as detection reagents to the arrays to which the corresponding binding reagents (mouse or rabbit antibodies) had been applied. Finally, the arrays were washed with assay buffer containing 5% BSA and subjected to the detection step by means of excitation and detection of the resulting fluorescence signals using the ZeptoREADER™ (see below).

3.4 Detection of the Fluorescence Signals from the Arrays of Measurement Areas

The fluorescence signals from the various arrays of measurement areas were measured sequentially in an automated way, using a ZeptoREADER™ (Zeptosens AG, Benkenstrasse 254, CH-4108 Witterswil). The principle measurement steps are as follows: For each array of measurement areas, the analytical platform according to the invention is adjusted for matching the resonance condition for in-coupling of light into the waveguiding tantalum pentoxide layer and for maximizing the excitation light available in the measurement areas. Then, for each array, images of the fluorescence signals from the corresponding array are generated, wherein the user can select different exposure times and the number of images to be generated. In the case of measurements for the present example, the excitation wavelength was 633 nm for excitation of Cy5 or Alexa Fluor 647 fluorescence labels and 532 nm for excitation of Cy3 fluorescence labels. The detection of the fluorescence light at the fluorescence wavelength of Cy5 or Alexa Fluor 647 is performed using a cooled camera, an interference filter (transmission 675 nm±25 nm, “red detection channel”) for suppression of scattered light being positioned in front of the lens of the camera. For detection of fluorescence light emanating from Cy3 fluorescence labels, excited at 532 nm, an interference filter with transmission at 572 nm±25 nm (“green detection channel”) is used. The fluorescence images generated are automatically stored on the disk of the control computer. Further details of the optical system (ZeptoREADER™) are described in the international patent application PCT/EP 01/10012, which is incorporated in its entirety in the present application.

3.5. Data Analysis

The fluorescence signal intensities corresponding to the relative concentration of the lysate sample analyte concentration from the measurement areas (spots) was determined using an image analysis software (ZeptoVIEW™, Pro 2.0 Release 2.0, Zeptosens AG, CH-4108 Witterswil) enabling an automated analysis of the fluorescence images from a multitude of arrays of measurement areas.

The raw data of the individual pixels of the camera correspond to a two-dimensional matrix of digitized measurement data, corresponding to the imaged area on the sensor platform. For data analysis, first a two-dimensional coordinate grid is superimposed on the image points (pixels) in such a way that the image fraction of each spot is contained in an individual two-dimensional grid element. Within this grid element, an adjustable, circular “area of interest” (AOI) with a user-definable radius is assigned to each spot. In this case, the spot diameter was set constant at 120 μm. The arithmetic mean of the pixel values (signal intensities) within a chosen analysis area is determined as the mean gross signal intensity for each spot.

The background signals are determined from the signal intensities measured between the spots. For this purpose, four additional circular areas (typically with the same radius as the analysis areas of the spots) are defined as analysis areas for background signal determination for each spot, which are preferably located in the center between adjacent spots. The mean background signal intensity is, for example, determined as the arithmetic mean of the pixel values (signal intensities) within a defined AOI for each of the four circular areas. The mean net signal intensity from the measurement areas (spots) is then calculated as the difference between the mean local gross and background signal intensities of the corresponding spots.

Referencing of the net signal intensities of all lysate sample spots is performed by means of reference spots (Cy5-BSA) of each array of measurement areas. For this purpose, an artificial reference spot signal intensity of each lysate sample spot position between two adjacent measured reference spots in a particular row is calculated by interpolation. Then, for each lysate sample spot, a referenced fluorescence intensity is calculated by division of the mean analyte spot net signal intensity by the mean value of the corresponding artificial reference spot net signal intensity This referencing method compensates for local differences in the available excitation light intensity along the direction perpendicular to the direction of light propagation, both within each microarray and between different microarrays.

For further analysis, the referenced fluorescence intensities (RFI), averaged from each duplicate spot pair as described above, were plotted as a function of the applied protein concentration of the respective tissue lysate, for each treatment and each antibody (data not shown). As an example, such dilution plots as calibration curves are shown for the m-actin measurement (FIG. 3). From the dilution plots, signals were normalized for the applied protein concentrations (of the 4 dilutions) applying best linear fits to the experimental data. Afterwards, the normalized signals were averaged. Consequently, each array field comprising 4 different dilutions of a cell lysate (8 spots) finally provides a normalized expression signal for each sample and antibody. The results are finally summarized as bar plot profiles, each bar representing the normalized expression signal (in RFI); error bars corresponding to the standard deviations of these normalized expression signals.

4. Results

4.1. Production of Tissue Lysate Arrays—Quality Control

The quality of spotted lysate arrays (e.g. number of missing spots, spot shape, spot morphology, relative protein concentration) was examined with an assay measuring the expression level of β-actin as a housekeeping protein. The measurement was performed in the green detection channel of the ZeptoREADER™ using a Cy3-labelled anti-β-actin antibody as a detection reagent, applied on arrays of measurement areas denoted as “Nondisclosed cell lysates in FIG. 1.

A good array quality of the produced chips was achieved:
No missing spots
Good spot shape
Good spot homogeneity

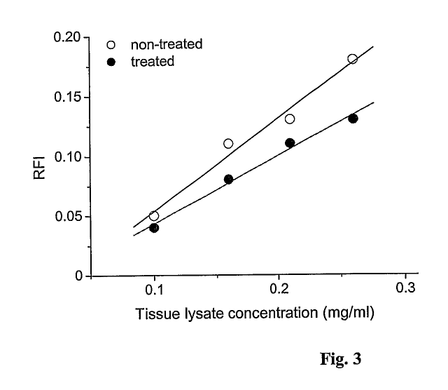

FIG. 2 shows on the left a fluorescence image (exposure time 5 sec, display range 0-20,000) after incubation with 1:500 diluted Cy3-anti-β-actin. The right side of FIG. 2 illustrates the layout (geometry) of the array (1=Non-disclosed, control cell lysate, 2=Non-disclosed, treated cell lysate, 3=Spotting buffer, 4=Non-treated colorectal cancer tissue lysate=“tumor lysate 1”, 5=Treated colorectal cancer tissue lysate=“tumor lysate 2”, 6=Empty; dilution of deposited sample increasing from left to right for each lysate, see also FIG. 1).

The spotting of cell/tissue lysates at four different dilutions allowed
to address the dynamic range of signal generation, and
to extract more accurate signals by using dose-dependent signal slopes instead of single point measurements (i.e. fluorescence intensities measured only for individual protein concentrations).

A clear decrease of fluorescence signal intensity with decreasing spotted tissue lysate protein concentrations could be observed for all applied lysates. As an example, a dilution plot with a linear dependence of the referenced fluorescence signals on the protein concentration in the deposited lysate samples, which was obtained by detection with the Cy3-anti-β-actin antibody for the non-treated and treated tumor tissue, is shown in FIG. 3. The data points always indicate the mean net fluorescence signals of 2 replicate spots per protein concentration (RFI=referenced fluorescence intensity).

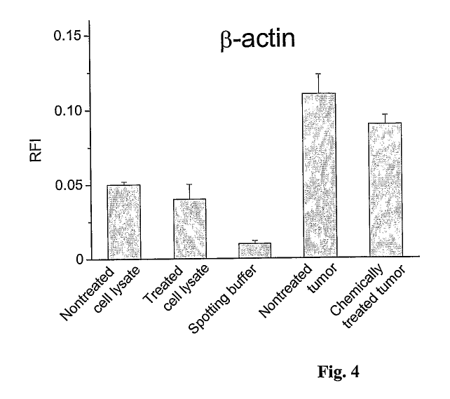

FIG. 4 shows a bar plot profile for the detection of β-actin for all spotted cell/tissue lysates, which was obtained from the dilution plots as described in section 3.5

For the “non-disclosed cell lysates”, fluorescence signal levels were of nearly comparable height, indicating that the different cell lysates were spotted at same concentration and that the treatment did not affect the level of β-actin. For the colon cancer tissue lysates, a slightly higher level of β-actin was detected for the treated sample compared to the non-treated one. Since the protein concentration of the two tissue lysates was equalized to the same level before preparing the spotting solutions, the obtained difference is probably due to an error in determining the right protein concentration from the standards (typical error within a range of 10-15%). Therefore, in the following, all analyte specific signals of the treated cancer lysate were corrected for this difference of β-actin signals.

4.2. Protein Expression Profiling of 14 Signaling Pathway Markers

One array of measurement areas was measured for each different antibody applied as specific binding reagent and protein to be detected in the deposited samples. In the following, the bar plot profile obtained from each measured microarray is shown for each protein analyte.

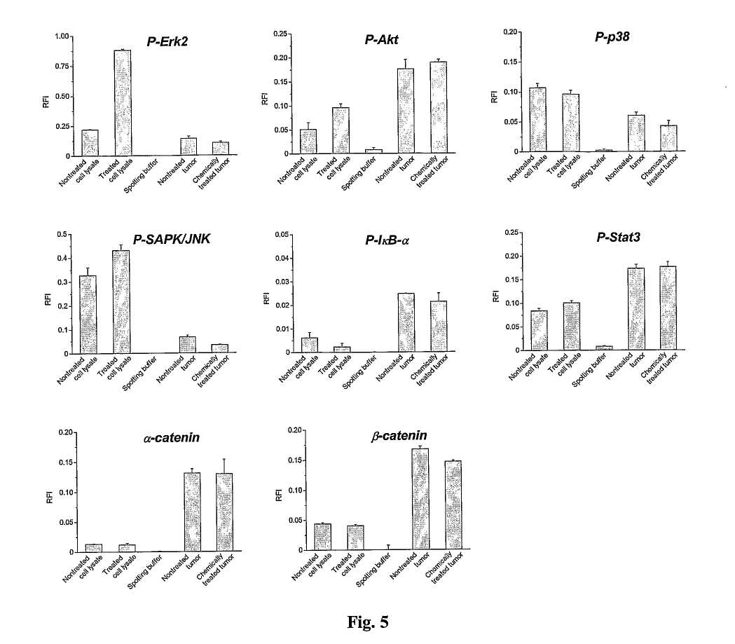

FIG. 5 shows the bar profiles for the detection of the pathway activation markers P-Erk2, P-Akt, P-p38, P-SAPK/JNK, P-IκB-α, P-Stat3, α-catenin, and β-catenin in the different samples.

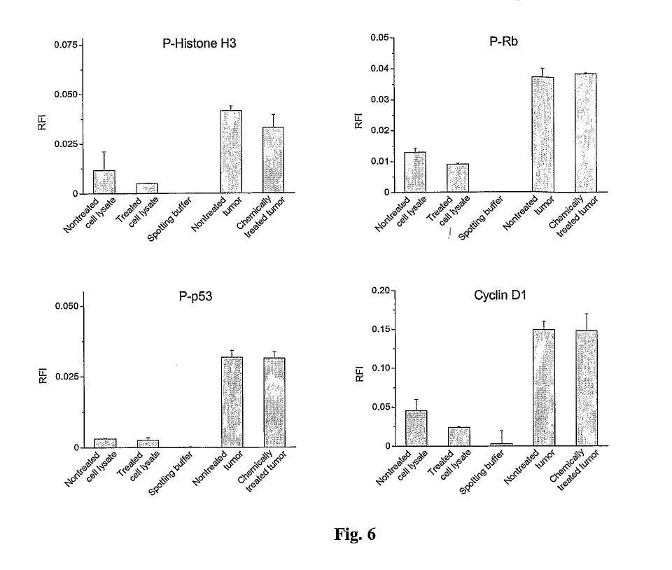

FIG. 6 shows the bar profiles for the detection of the proliferation markers P-Histone H3, P—Rb, P-p53, and Cyclin D1in the different samples.

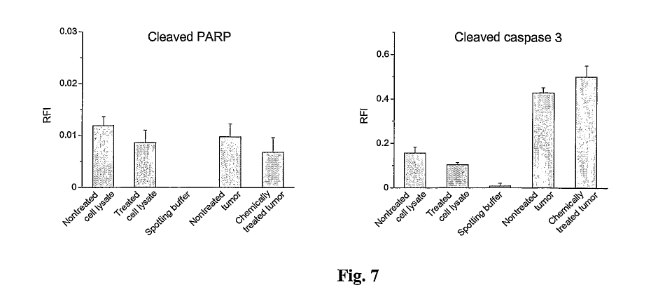

FIG. 7 shows the bar profiles for the detection of apoptosis markers (cleaved PARP, cleaved caspase 3) in the different samples.

4.3. Differential Protein Expression Profile/Summary of the Results

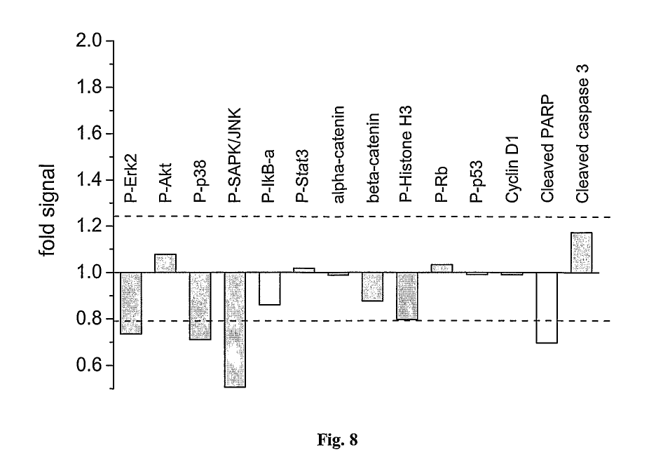

FIG. 8 summarizes the results obtained for the two differently treated colon cancer tissue lysates profiled with the 14 different marker antibodies, for binding to and detection of the 14 different protein analytes. “Fold signal” changes were calculated as ratios of the signals of treated lysate sample over signals of control lysate sample and thus represent a differential expression profile of the proteins of interest. The broken lines indicate the limits of significant up- and down-regulation of protein expression:
Limit for up-regulation=1.24
Limit for down-regulation=0.81

These limits were determined from a larger set of comparable study experiments, which typically reveal CV's of fold signal changes in the range of 7-14% (mean 10%) from duplicate antibody experiments. In the experiments here, the typical coefficient of variation, averaged over the mean expression signals (from 8 spot replicates each) of all 14 single antibody measurements, was in a range of 2-20% (mean CV=8%).

Signal values above/below these limit lines are indicative for significantly altered protein expression levels upon treatment. As visible from the plot, no significant up-regulation could be observed for any of the investigated signaling marker proteins. Prominent down-regulation could be observed only for one protein, P-SAPK/JNK of the group of pathway activation markers (signal level at 0.50). Two other pathway activation markers showed minor down regulation near the limits of significance: P-Erk2 (signal level 0.74) and P-p38 (signal level 0.71). All other analytes showed no significant signal changes.

1. A method for generating qualitative and/or quantitative protein expression profiles of one or more populations of cells comprising:
generating lysates of one or more populations of cells, the lysates comprising a plurality of proteins expressed by the respective cell populations,
providing an essentially planar solid support,
depositing at discrete sites small quantities of the cell lysates as deposited samples, in diluted or undiluted form directly on said solid support or on an adhesion-promoting layer applied on said solid support, thereby creating one or more one- or two-dimensional arrays of discrete measurement areas on said solid support,
applying a number of binding reagents as specific binding partners for the proteins contained in cell lysates in discrete measurement areas and to be detected and, if adequate, one or more detection reagents on said one or more arrays of measurement areas, the binding reagents and the detection reagents being applied sequentially or in a single addition-step, after binding of the detection reagents to the binding reagents, to the one or more arrays of discrete measurement areas, and
measuring and recording optical signals emanating from said one or more arrays of discrete measurement areas in a locally resolved manner,
wherein said essentially planar solid support is non-porous and optionally comprises applied adhesion-promoting layer having a thickness of less than 1 μm.

2. A method for generating qualitative and/or quantitative differential protein expression profiles of two or more populations of cells comprising:
generating a first lysate of a population of cells, the lysate comprising a plurality of proteins expressed by the respective cell population,
generating second or more lysates of further populations of cells, the lysates comprising pluralities of proteins expressed by the respective cell population,
providing an essentially planar solid support,
depositing at discrete sites small quantities of the cell lysates as deposited samples, in diluted or undiluted form directly on said solid support or on an adhesion-promoting layer applied on said solid support, thereby creating one or more one- or two-dimensional arrays of discrete measurement areas on said solid support,
applying a number of binding reagents as specific binding partners for the proteins contained in cell lysates in discrete measurement areas and to be detected and, if adequate, one or more detection reagents on said one or more arrays of measurement areas, the binding reagents and the detection reagents being applied sequentially or in a single addition-step, after binding of the detection reagents to the binding reagents, to the one or more arrays of discrete measurement areas, and
measuring and recording a first group of optical signals emanating from the measurement areas created by deposition of small quantities of the first lysate, in diluted or undiluted form, in a locally resolved manner,
measuring and recording second or more groups of optical signals emanating from the measurement areas created by deposition of small quantities of the second or more lysates, in diluted or undiluted form, in a locally resolved manner,
comparing the measured values of the first group of optical signals with the values of the second or more groups of optical signals,
wherein said essentially planar solid support is non-porous and optionally comprises an applied adhesion-promoting layer having a thickness of less than 1 μm.

3. A method according to claim 1, wherein different binding reagents as specific binding partners for different proteins are applied on different arrays for each different protein to be detected.

4. A method according to claim 1, wherein different proteins are detected in a common array by applying different distinguishable detection reagents on said array, the number of different proteins to be detected corresponding to the number of different distinguishable labels applied.

5. A method according to claim 1, wherein a plurality of different proteins is detected in multiple arrays of measurement areas by applying different binding reagents as specific binding partners for different proteins on different arrays for the detection of different proteins and/or different distinguishable detection reagents on the arrays of measurement areas.

6. A method according to claim 1, wherein different lysates are generated from unrelated cell populations.

7. A method according to claim 1, wherein different lysates are generated from different cell sub-populations that have been obtained from a common cell population.

8. A method according to claim 7, wherein different lysates are generated from different cell sub-populations that have been obtained from a common cell population at different points in time.

9. A method according to claim 7, wherein different lysates are generated from different cell sub-populations that have been obtained from a common cell population and then treated or stimulated with different reagents and/or exposed to different cultivation conditions.

10. A method according to claim 1, wherein different lysates are generated from diseased and healthy cell populations.

11. A method according to claim 1, wherein healthy or diseased and/or treated or untreated and/or stimulated cell populations from which the lysates have been generated, have been derived from the group consisting of prokaryotic cells, eukaryotic cells, plant cells, human or animal tissue, and plant tissue.

12. A method according to claim 1, wherein the lysates, in diluted or undiluted form, that are deposited at discrete sites on the solid support or on an adhesion-promoting layer on said solid support have the same relative molecular compositions of the proteins to be detected therein as the cell populations from which the lysates have been generated.

13. A method according to claim 1, wherein the lysates contain added known concentrations of compounds (as standards) similar to the analytes to be determined as additives.

14. A method according to claim 1, wherein the material deposited in a single measurement area corresponds to the protein content of less than 1000 cells.

15. A method according to claim 1, wherein multiple arrays of measurement areas are arranged in an identical geometry of the deposition sites of the diluted or undiluted cell lysates, a similar position with respect to rows and column of a measurement area in two different arrays corresponding to deposited amounts from the same (diluted or undiluted) cell lysate deposited therein.

16. A method according to claim 1, wherein an adhesion-promoting layer applied on the solid support has a thickness of less than 200 nm.

17. A method according to claim 16, wherein said adhesion-promoting layer comprises compounds selected from the group consisting of silanes, functionalized silanes, epoxides, functionalized, charged or polar polymers and “self-organized passive or functionalized mono- or multi-layers”, thiols, alkyl phosphates and alkyl phosphonates, and multi-functional block copolymers.

18. A method according to claim 16, wherein the samples are deposited laterally selectively in discrete measurement areas, directly on the solid support or on an adhesion-promoting layer deposited thereon, by means of a method selected from the group of methods consisting of ink jet spotting, mechanical spotting by pen, pin or capillary, “micro contact printing”, fluidic contacting of the measurement areas with the samples through their supply in parallel or crossed micro channels, with application of pressure differences or electrical or electromagnetic potentials, and photochemical or photolithographic immobilization methods.

19. A method according to claim 1, wherein regions between the discrete measurement areas are passivated in order to minimize nonspecific binding of binding and/or detection reagents, i.e. that compounds which are chemically neutral (i.e. nonbinding) towards the analytes (i.e. proteins) and the other contents of the deposited samples and the binding reagents and, if adequate, towards the detection reagents are deposited between the laterally separated measurement areas.

20. A method according to claim 1, wherein the proteins which are to be detected and are contained in the diluted or undiluted lysates deposited in discrete measurement areas are compounds selected from the group consisting of proteins comprising cytosolic, nuclear and membrane proteins, secreted proteins in body fluids, post-translationally modified proteins.

21. A method according to claim 1, wherein the proteins which are to be detected and are contained in the diluted or undiluted lysates deposited in discrete measurement areas are distinguished in the step of binding added specific binding reagents and, if adequate, detection reagents, added sequentially or in a single addition step, after binding of the detection reagents to the binding reagents, according their occurrence in phosphorylated and/or nonphosphorylated form and/or glycosylated and/or nonglycosylated form and/or methylated and/or non-methylated form and/or acetylated and/or non-acetylated form contained in the diluted or undiluted deposited lysates to be analyzed.

22. A method according to claim 1, wherein the proteins which are to be detected and are contained in the diluted or undiluted lysates deposited in discrete measurement areas are not distinguished in the step of binding added specific binding reagents and, if adequate, detection reagents, added sequentially or in a single addition step, after binding of the detection reagents to the binding reagents, between their occurrence in phosphorylated or nonphosphorylated form and/or glycosylated or nonglycosylated form and/or methylated or non-methylated form and/or acetylated or non-acetylated form contained in the diluted or undiluted deposited lysates to be analyzed.

23. A method according to claim 1, wherein the material of the essentially planar solid support being in physical contact with the generated measurement areas either directly or mediated by an adhesion promoting layer is essentially optically transparent.

24. A method according to claim 16, wherein the material of an adhesion layer applied on the solid support is essentially optically transparent.

25. A method according to claim 1, wherein the material of the essentially optically transparent solid support comprises a material from the group consisting of moldable, sprayable or millable plastics, metals, metal oxides, and silicates.

26. A method according to claim 1-25, wherein probing light from one or more polychromatic or monochromatic light sources is directed towards one or more measurement areas in one or more arrays of measurement areas and optical signals emanating from said one or more arrays of measurement areas and/or changes in these optical signals are measured and recorded.

27. A method according to claim 26, wherein the probing light is delivered in an epi-illumination configuration.

28. A method according to claim 26, wherein the probing light is delivered in a trans-illumination configuration.

29. A method according to claim 1, wherein the detection of one or more proteins in discrete measurement areas is based on the detection of the intensities or changes in the intensities of one or more luminescences.

30. A method according to claim 1, wherein the detection of one or more proteins in discrete measurement areas is based on the detection of changes in the refractive index on said measurement areas or within a distance of less than 1 μm from these measurement areas.

31. A method according to claim 30, wherein the detection of changes in the refractive index on said measurement areas or within a distance of less than 1 μm from these measurement areas is based on detection of changes in the pattern of interferences of light emanating from the planar solid support in the regions of the measurement areas generated on the solid support with light emanating from planes of interfaces to materials of different refractive index, caused by changes of the phase differences between the light emanating from said interfaces and the light emanating from the regions of the measurement areas due to binding or desorption or displacement of applied specific binding partners, and wherein the interference light emanating from the different regions is measured in a locally and, if adequate, spectrally resolved manner.

32. A method according to claim 1, wherein the solid support is provided with a thin metal layer, optionally with a thickness between 20 nm and 200 nm, which is directly or mediated by an adhesion-promoting layer in contact with the measurement areas, and the detection of changes in the refractive index on said measurement areas or within a distance of less than 1 μm from these measurement areas is based on detection of changes in the conditions for generating a surface plasmon resonance in said metal layer.

33. A method according to claim 1, wherein the solid support comprises a continuous optical waveguide or an optical waveguide divided into individual waveguiding areas.

34. A method according to claim 33, wherein the optical waveguide is an optical film waveguide with a first essentially optically transparent layer (a) facing the surface carrying the discrete measurement areas on a second essentially optically transparent layer (b) with a refractive index lower than that of layer (a).

35. A method according to claim 34, wherein, for the in-coupling of probing light into the optically transparent layer (a), this layer is in optical contact with one or more optical in-coupling elements selected from the group consisting of prism couplers, evanescent couplers with combined optical waveguides with overlapping evanescent fields, butt-end couplers with focusing lenses arranged in front of one face of the waveguiding layer, and grating couplers.

36. A method according to claim 35, wherein the probing light is in-coupled into the optically transparent layer (a) using one or more grating structures (c) which are featured in the optically transparent layer (a).

37. A method according to claim 34, wherein light guided in the optically transparent layer (a) is out-coupled using one or more grating structures (c′) which are featured in the optically transparent layer (a).

38. A method according to claim 34, wherein the detection of proteins in the measurement areas takes place via a grating structure formed in the layer (a) of an optical film waveguide based on changes in the resonance conditions for the in-coupling of probing light into layer (a) of a solid support formed as film waveguide or for out-coupling of light guided in layer (a), these changes resulting from binding of binding reagents and/or further detection reagents to proteins contained in the measurement areas.

39. A method according to claim 34, wherein said optical waveguide is designed as an optical film waveguide with a first optically transparent layer (a) on a second optically transparent layer (b) with lower refractive index than layer (a), wherein probing light is further in-coupled into the optically transparent layer (a) with the aid of one or more grating structures, which are featured in the optically transparent layer (a), and delivered as a guided wave to measurement areas (d) located thereon, and wherein the luminescence of molecules capable of luminescence, generated in the evanescent field of said guided wave, is further determined using one or more detectors, and the relative amount of proteins contained in the measurement areas is determined from the intensity of these luminescence signals.

40. A method according to claim 39, wherein luminescences are generated upon excitation of detection reagents associated with binding reagents that have specifically bound to proteins to be detected in the measurement areas, and wherein the detection reagents comprise luminescent dyes or luminescent nanoparticles used as luminescence labels, which can be excited and emit at wavelengths between 300 nm and 1100 nm.

41. A method according to claim 40, wherein different distinguishable detection reagents feature different emission wavelengths and/or different emission lifetimes.

42. A method according to claim 1, wherein the probing light is delivered in pulses with a duration between 1 fs and 10 minutes and the emission light from the measurement areas is measured in a time-resolved manner.

43. An analytical platform for optical signal read-out and for generating qualitative and/or quantitative protein expression profiles of one or more populations of cells comprising:
an essentially planar solid support,
one or more one- or two-dimensional arrays of discrete measurement areas on said solid support, said arrays being generated by deposition of small quantities of cell lysates, in diluted or undiluted form, at discrete sites directly on said solid support or on an adhesion-promoting layer applied on the solid support before, the cell lysates originating from one or more populations of cells and containing a plurality of proteins expressed by these cell populations,
wherein said essentially planar solid support is non-porous and comprises an optionally applied adhesion-promoting layer having a thickness of less than 1 μm.

44. An analytical platform for optical signal read-out and for generating qualitative and/or quantitative differential protein expression profiles of one or more populations of cells comprising:
an essentially planar solid support,
one or more one- or two-dimensional arrays of discrete measurement areas on said solid support, said arrays being generated by deposition of small quantities of two or more cell lysates, in diluted or undiluted form, at discrete sites directly on said solid support or on an adhesion-promoting layer applied on the solid support before, the cell lysates originating from two or more populations of cells and containing a plurality of proteins expressed by these cell populations,
wherein said essentially planar solid support is non-porous and comprises an optionally applied adhesion-promoting layer having a thickness of less than 1 μm.

45. An analytical platform according to claim 43, comprising different deposited lysates generated from unrelated cell populations.

46. An analytical platform according to claim 43, comprising different deposited lysates generated from different cell sub-populations that have been obtained from a common cell population.

47. An analytical platform according to claim 46, comprising different deposited lysates generated from different cell sub-populations that have been obtained from a common cell population at different points in time.

48. An analytical platform according to claim 46, comprising different deposited lysates generated from different cell sub-populations that have been obtained from a common cell population and then treated or stimulated with different reagents and/or exposed to different cultivation conditions.

49. An analytical platform according to claim 43, comprising different deposited lysates generated from diseased and healthy cell populations.

50. An analytical platform according to claim 43, comprising deposited lysates generated from healthy or diseased and/or treated or untreated and/or stimulated cell populations selected from the group consisting of prokaryotic cells, and eukaryotic cells.

51. An analytical platform according to claim 43, wherein the lysates, in diluted or undiluted form, that are deposited at discrete sites on the solid support or on an adhesion-promoting layer on said solid support have the same relative molecular compositions of the proteins to be detected therein as the cell populations from which the lysates have been generated.

52. An analytical platform according to claim 43, wherein the deposited lysates have been subjected to no further sample treatment steps than filtration and/or fractionation and/or dilution.

53. An analytical platform according to claim 43, wherein the material deposited in a single measurement area corresponds to the protein content of less than 1000 cells.

54. An analytical platform according to claim 43, wherein multiple arrays of measurement areas are arranged in an identical geometry of the deposition sites of the diluted or undiluted cell lysates, a similar position with respect to rows and column of a measurement area in two different arrays corresponding to deposited amounts from the same (diluted or undiluted) cell lysate deposited therein.

55. An analytical platform according to claim 43, wherein an adhesion-promoting layer applied on the solid support has a thickness of less than 200 nm.

56. An analytical platform according to claim 55, wherein said adhesion-promoting layer comprises compounds selected from the group consisting of silanes, functionalized silanes, epoxides, functionalized, charged or polar polymers and “self-organized passive or functionalized mono- or multi-layers”, thiols, alkyl phosphates and alkyl phosphonates, and multi-functional block copolymers.

57. An analytical platform according to claim 43, wherein regions between the discrete measurement areas are passivated in order to minimize nonspecific binding of tracer compounds.

58. An analytical platform according to claim 43, wherein the proteins which are to be detected and are contained in the diluted or undiluted lysates deposited in discrete measurement areas are compounds selected from the group consisting of proteins comprising cytosolic, nuclear and membrane proteins, secreted proteins in body fluids, and post-translationally modified proteins.

59. An analytical platform according to claim 43, wherein the material of the essentially planar solid support being in physical contact with the generated measurement areas either directly or mediated by an adhesion promoting layer is essentially optically transparent.

60. An analytical platform according to claim 43, wherein the material of an adhesion layer applied on the solid support is essentially optically transparent.

61. An analytical platform according to claim 43, wherein the material of the essentially optically transparent solid support comprises a material selected from the group consisting of moldable, sprayable or millable plastics, metals, metal oxides, and silicates.

62. An analytical platform according to claim 43, wherein the solid support is provided with a thin metal layer, and optionally with a thickness between 20 nm and 200 nm, which is directly or mediated by an adhesion-promoting layer in contact with the measurement areas, the platform being operable for generating a surface plasmon resonance in said metal layer.

63. An analytical platform according to claim 43, wherein the solid support comprises a continuous optical waveguide or an optical waveguide divided into individual waveguiding areas.

64. An analytical platform according to claim 63, wherein the optical waveguide is an optical film waveguide with a first essentially optically transparent layer (a) facing the surface carrying the discrete measurement areas on a second essentially optically transparent layer (b) with a refractive index lower than that of layer (a).

65. An analytical platform according to claim 64, wherein, for the in-coupling of probing light into the optically transparent layer (a), this layer is in optical contact with one or more optical in-coupling elements selected from the group consisting of prism couplers, evanescent couplers with combined optical waveguides with overlapping evanescent fields, butt-end couplers with focusing lenses arranged in front of one face of the waveguiding layer, and grating couplers.

66. An analytical platform according to claim 65, wherein one or more grating structures (c) are featured in the optically transparent layer (a) for allowing in-coupling of probing light into the optically transparent layer (a).

67. An analytical platform according to claim 64, wherein one or more grating structures (c′) are featured in the optically transparent layer (a), which allow out-coupling of light guided in the optically transparent layer (a).

68. An analytical platform according to claim 64, wherein said optical waveguide is designed as an optical film waveguide with a first optically transparent layer (a) on a second optically transparent layer (b) with lower refractive index than layer (a), and wherein the analytical platform is operable of in-coupling probing light into the optically transparent layer (a) with the aid of one or more grating structures, which are featured in the optically transparent layer (a), delivering said probing light as a guided wave to measurement areas (d), and exciting luminescence of molecules capable of luminescence in the evanescent field of said guided wave.

69. A method of using the method according to claim 1 for quantitative and/or qualitative analyses for the determination of proteins and their modified forms in screening methods in pharmaceutical research, combinatorial chemistry, clinical and pre-clinical development, for real-time binding studies and the determination of kinetic parameters in affinity screening and in research, for the measurement of protein-DNA interactions, for the determination of control mechanisms for the protein (bio)synthesis, for the screening of biological and chemical marker compounds, for patient stratification in pharmaceutical product development and for the therapeutic drug selection.
